(19) United States
(12) Patent Application Publication
(10) Pub. No.: US 2013/0296604 A1
(43) Pub. Date: Nov. 7, 2013

(54) METHOD OF MAKING AND USING NANO-BIMETALLIC CATALYST TO PRODUCE ADIPIC ACID

(71) Applicants: Ahmad S. ALSHAMMARI, Riyadh (SA); Abdulaziz A. BAGABAS, Riyadh (SA); Angela KÖCKRITZ, Berlin (DE); Venkata Narayana KALEVARU, Berlin (DE); Andreas MARTIN, Berlin (DE)

(72) Inventors: Ahmad S. ALSHAMMARI, Riyadh (SA); Abdulaziz A. BAGABAS, Riyadh (SA); Angela KÖCKRITZ, Berlin (DE); Venkata Narayana KALEVARU, Berlin (DE); Andreas MARTIN, Berlin (DE)

(73) Assignee: King Absulaziz City for Science and Technology (KACST), Riyadh (SA)

(21) Appl. No.: 13/464,904

(22) Filed: May 4, 2012

(51) Int. Cl. B01J 23/52; B01J 23/44; C07C 51/31; B01J 23/40; B01J 21/06; B82Y 40/00; B82Y 30/00

(52) U.S. Cl. 562/543; 502/330; 502/347; 977/810; 977/896; 977/902

(57) ABSTRACT

A bimetallic catalyst supported on a transition metal oxide is described. A method to make and use the bimetallic catalyst is also described. A method for preparing supported bimetallic catalysts of coinage group metal and a combination of a coinage group metal and a platinum metal group is described. A method for direct synthesis of adipic acid (AA) adopting green catalytic oxidation route of cyclohexane (CH) using the bimetallic catalysts is described. The reaction to convert CH into AA in the presence of bimetallic catalyst is carried in an autoclave in the temperature range of 25 to 300° C. The CH conversion was over 21% with AA selectivity of 34% and ca. 63% selectivity of cyclohexanone and cyclohexanol together over Au—Pd/TiO₂ bimetallic catalyst.

### FIELD OF INVENTION

The instant application relates to the production of Adipic acid (AA) from cyclohexane (CH) using a nano-bimetallic catalyst. The nano-bimetallic catalyst is specifically a combination of a coinage group metal and a platinum group metal.

### BACKGROUND

The selective oxidation of cyclohexane (CH) to adipic acid (AA) is an industrially important reaction for manufacturing various valuable materials such as polyamides, polyurethanes, polyesters, plasticizers, intermediates for pharmaceuticals and insecticides etc. AA is also used in medicine and food industry for different applications.

The current, commercial production process of AA is a two-step process. The first step is the formation of cyclohexanone and cyclohexanol (i.e., KA—K for ketone and A for alcohol) at around 150° C. and at 10-20 bar of air using a cobalt or a manganese catalyst. KA can also be obtained by phenol hydrogenation, as shown in FIG. 1. The second step is an oxidation of KA into AA using nitric acid. This method is environmentally harmful, expensive, and energy-demanding. The use of nitric oxide generates and liberates nitrogen oxide gases (NOₓ) that are harmful to the environment. On the other hand recycling >90% of un-reacted CH increases the production cost and the energy demand. Besides the commercial process, there are other routes for producing AA. For examples, AA can be obtained by direct oxidation of CH using hydrogen peroxide, by carbonylation of butadiene, by dimerization of methyl acrylate, or fermentation of glucose (FIG. 1).

Even though some of the above processes, shown in FIG. 1, are being practiced commercially, most of them suffer from high costs due to multi-step operations and handling large waste disposal. On the other hand, some process options for AA production without the use of HNO₃ were also proposed by various research groups in different patents (e.g. GB 1304 855 (1973) and U.S. Pat. No. 3390174 (1968). Nevertheless, these approaches gave only poor selectivities (S=30-50%) of AA. Another problem of most of these processes is the use of soluble homogeneous catalysts, which leach out during the reaction and pose difficulty for separation after the reaction.

The use of heterogeneous, solid catalyst in the direct oxidation of CH to AA is also known from the prior art. For example, F. T. Starzyk et al. have applied iron phthalocyanine encapsulated in Y-zeolite as a catalyst for the direct oxidation of CH to AA. However, this process strongly suffers from much longer induction periods, i.e. the catalyst requires about 300 h to reach CH conversion of ca. 35% and needs 600 h to get higher amounts of adipic acid in the product stream, which makes the process commercially unattractive.

Furthermore, efforts were also made by various researchers to use gold-based catalysts for the direct oxidation of CH to AA, but to the best of our knowledge, all such attempts failed until now. For instance, various gold catalysts such as Au/graphite, Au/MCM-41, Au/SBA-15, Au on CeO₂, SiO₂ and Al₂O₃ supports were applied for the said reaction, which gave only cyclohexanol and cyclohexanone as major products without any adipic acid in the product stream. Using such catalyst systems, the conversion of CH was varied in the range from 6 to 20%. However, the selectivity of both cyclohexanol and cyclohexanone products together were found to be in the range of 17 to 90% depending on the catalyst system used and the reaction conditions applied.

Therefore, there is need to develop a novel, environmentally benign production of AA.

### SUMMARY OF THE INVENTION

The disclosure describes a catalyst, a method of making a nano-bimetallic catalyst (may be called bimetallic catalyst as well in the specification) and a single step process of using the nano-bimetallic catalyst to produce AA from CH.

In one embodiment, a nano-bimetallic catalyst comprising mainly two metals; one from the coinage metal group and the other one from the platinum metal group, supported on a transition or rare earth metal oxide is disclosed.

In one embodiment, a method of making a supported nano-bimetallic catalyst is described. In one embodiment, a suitable precursor is used to make the aqueous bimetallic solution. In another embodiment, reducing of the bimetallic solution using an aqueous solution of tannic acid and sodium citrate as appropriate reductants and capping agents is described. In further embodiment, impregnation of the above colloidal solution onto the catalyst support, followed by evaporation of water until dryness is described. In another embodiment, oven drying and calcination under suitable conditions/atmosphere of the catalyst is described. This new preparation method provides an active and selective catalyst. This simple method of preparation also allows obtaining a catalyst with improved performance.

In one embodiment, a direct method for producing AA in a single step with acceptable selectivity from the direct oxidation of CH using effective and potential catalyst compositions is described. In one embodiment, a method for the preparation of AA by the one-pot oxidation of CH, which comprises reacting mixture of CH with O₂ in presence of a solid nano-bimetallic catalyst is described. The nano-bimetallic catalyst is a supported catalyst on a transition metal oxide such as TiO₂. More particularly, the said reaction was carried out at a reaction temperature in the range of 50 to 250° C. in a 100-ml autoclave.

In one embodiment, the influence of the nature of the second metal on the catalytic performance is described. In another embodiment, a direct oxidation of CH to AA in one step by O₂, as an oxidant, is described as an economic, environmentally friendly approach.

In one embodiment, a catalytic oxidation of cyclohexane using gold bimetallic catalyst is described and shown in the following chemical equation:

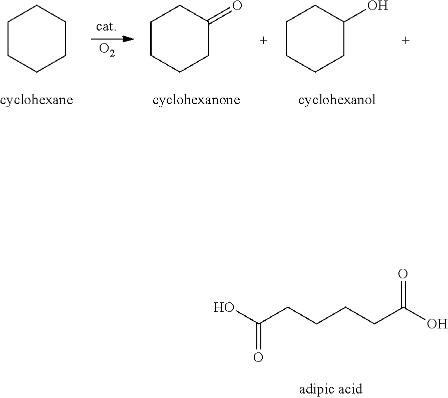

The catalyst and method of making the catalyst and method of using the catalyst disclosed herein may be implemented in any means for achieving various aspects, and may be executed manually. Other features will be apparent from the accompanying drawings and from the detailed description that follows.

### BRIEF DESCRIPTION OF DRAWING

Example embodiments are illustrated by way of example and no limitation in the tables and in the accompanying figures, like references indicate similar elements and in which:

FIG. 1 describes the summary of the different pathways for AA production.

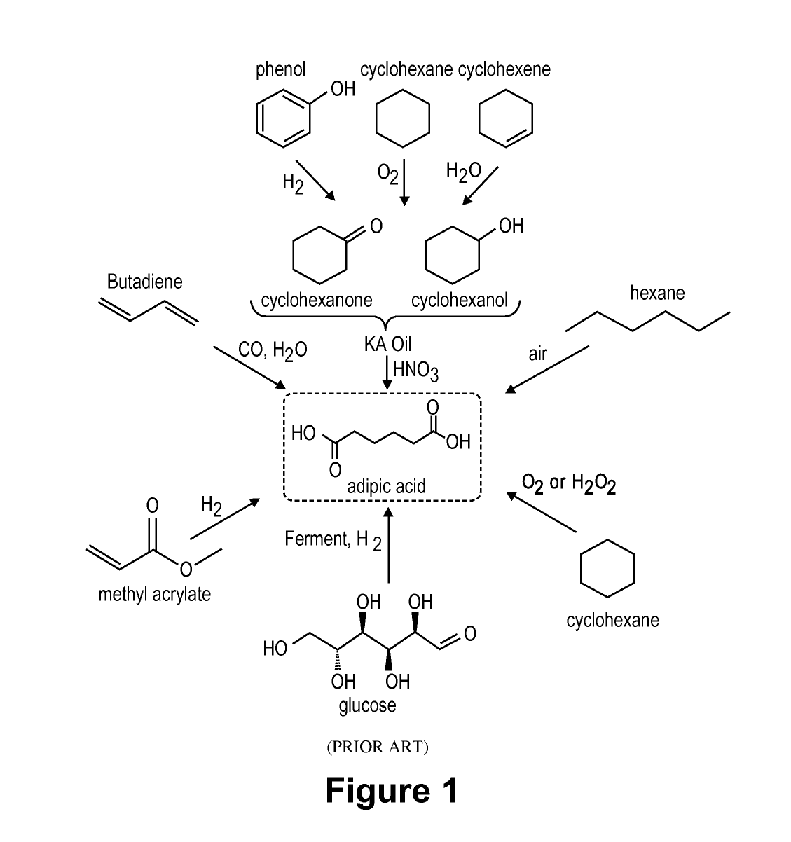

Other features of the present embodiments will be apparent from the accompanying drawings and from the detailed description that follows.

### DETAILED DESCRIPTION

A bimetallic catalyst, a method of synthesizing a novel bimetallic catalyst and utilizing the novel bimetallic catalyst to increase the production of AA from CH are disclosed. Although the present embodiments have been described with reference to specific example embodiments, it will be evident that various modifications and changes may be made to these embodiments without departing from the broader spirit and scope of the various embodiments.

### Bimetallic Catalyst and Method of Making the Bimetallic Catalyst

A bimetallic catalyst may comprise of two metals, one from coinage metal group, copper (Cu), silver (Ag), or gold (Au), and the other from platinum metal group, ruthenium (Ru), rhodium (Rh), palladium (Pd), osmium (Os), iridium (Ir), and platinum (Pt), supported on a transition or rare earth metal oxide such as titanium dioxide is described. In particular, catalyst Au/TiO₂, Pd/TiO₂ and AuPd/TiO₂ are being disclosed.

The method of making the catalyst involves several steps that are described below.

Step 1: A one-step chemical reduction of HAuCl₄-M colloids (M=PdCl₂, AgNO₃) was carried out to prepare Au-Pd and Au-Ag bimetallic colloidal systems. In the first step, a first solution was prepared dissolving tetrachloroauric acid (HAuCl₄.3H₂O, Lab-prepared, 0.06 g) and potassium carbonate (K₂CO₃, Aldrich, 0.1 g) in distilled water (600 mL). In the second step, a second solution of an aqueous palladium chloride (PdCl₂) solution (M) was prepared by dissolving required amount of PdCl₂ in 10 mL distilled water. This second solution was heated to 50° C. for 10 minutes and a few drops of hydrochloric acid (HCl) were added to completely dissolve the PdCl₂ precursor. The second solution was then mixed to the coinage group chloride solution or gold chloride solution (first solution) to form a bimetallic solution (third solution). A reduction reaction of bimetallic solution using a mixture of 1% tannic acid (reducing agent) and 1% of sodium citrate (stabilizer) by stifling (1000 rpm) at 60° C. was performed to produce a colloidal solution of two metals. In a similar way, Au—Ag bimetallic colloidal solution (fourth solution) was also prepared.

Step 2: In this step, the above-prepared colloidal solution of metals (step 1) was further mixed with a catalyst support (e.g. TiO₂) in powder form by stirring to result in a slurry of the metals colloid and the catalyst support.

Step 3: The slurry was vigorously stirred for another 2 hours at room temperature and then the excess solvent was removed on a rotary evaporator. This step produced a solid contain the colloidal metal nanoparticles impregnated onto the catalyst support. The solid thus obtained was washed three times with water. In one embodiment, the resultant solid catalyst is heated in a calcining atmosphere at a temperature in the range of 250° C. to 450° C., for a period of 3 to 20 hours. In another example, the solid catalyst is then oven dried at 120° C. for 16 h. The oven dried sample was finally calcined at 350° C. for 5 h in air.

The calcination can be done in different atmospheres, which include inert gas (N₂, He or Ar), air and reducing gas (H₂,); preferably air is used at a flow rate of 3-10 l/h.

The preparation of the present catalyst also involves the use of various sources of reducing agents. These reducing agents may include at least one of citric acid, sodium citrate, ascorbic acid, sodium thiocyanate, sodium borohydride, tannic acid, tartaric acid, oxalic acid, salts of the same or the like. This novel preparation method allows providing an active and selective catalyst. This simple method of preparation also allows obtaining a catalyst with good performance.

Using the same procedure, other bimetallic catalysts were also prepared in a similar way. The support used was TiO₂. Each metal was always fixed constant at 1 wt %.

### Method of Using the Bimetallic Catalyst to Make AA

The main focus of the present disclosure to illustrate the direct oxidation of CH to AA may be carried out using bimetallic catalysts in a stainless steel autoclave under the conditions mentioned below.

In the following examples, the conversion, yield and selectivity based on CH are illustrated using the following formula and shown in tables below:

Conversion (%)=A/B×100

where A is the number of moles reacted CH, and
B is the number of moles of CH fed to the reaction zone.

Yield (%)=C/B×100
where C is the number of moles AA obtained

Selectivity (%)=C/A×100

According to the method of using the catalyst to make AA, the starting compounds are cyclic paraffin's, referring to ring composed of 3 to 8 carbon atoms. “Oxidation” refers to the process of converting hydrocarbon moiety into oxidized products such as aldehydes, ketones and carboxylic acids in one step using a catalyst under oxygen. The reaction pressure may be atmospheric, sub-atmospheric or super-atmospheric. Preferably the pressure is in the range from atmospheric to 100 bars. Cyclohexane is oxidized by oxygen, air, or a mixture of oxygen and an inert gas diluent such as nitrogen, helium, argon, and neon. A method for preparing carboxylic acids by the selective oxidation of a cyclic paraffin at a reaction temperature comprising in the range of 50 to 250° C. using a nano-bimetallic catalyst in an autoclave is performed.

According to the invention, O₂ (air) was supplied to an autoclave containing a supported bimetallic catalyst (nano-bimetallic catalyst). The liquid feed in particular CH, tert-butyl hydroperoxide (TBHP) and solvent (e.g. acetonitrile) were mixed. Furthermore, the invention provides a method wherein the molar concentration of CH was preferably in the range from 2 to 20%. The mole ratio of solvent to CH was in the range of 4 to 20. The mole ratio of CH to TBHP was in the range of 10 to 60. The pressure of O₂ was in the range from atmospheric to less than 50 bar.

In one embodiment, the solvent used in the reaction may be selected from the group consisting of water, acetonitrile, benzene and any other organic solvent, which is inert under the conditions applied. The stifling speed of reaction mixture was varied in the range of 300 to 2000 rpm.

The reaction was carried out in an electrically heated stainless steel Parr autoclave. The liquid feed (i.e. CH, solvent, and TBHP) was placed in an autoclave in desired amounts. In a typical experiment, 400 milligrams of nano-bimetallic catalyst was added into the mixture, desired stirring speed of the reaction mixture was set and then the pressure of O₂ was also set appropriately. Then the temperature was raised gradually to the desired one and the reaction was performed. At the end of the reaction, the solid catalyst was separated by centrifugation. The products were analyzed by gas chromatography, equipped with flame-ionized detector (FID). Some selected experiments at ambient pressure were also performed using a glass reactor in a similar way as described above.

In particular, the AuPd/TiO₂ catalyst showed good potentiality and exhibited good conversion of CH (21%) with good selectivity of AA (34.5%). Thus, an acceptable yield of AA was successfully achieved.

### Catalyst Comparison Testing

The following paragraph illustrates the procedure for catalyst testing for the present reaction carried out according to the invention.

Activity tests were carried out under pressure using a Parr autoclave according to the procedure as follows. The reaction mixture comprised of 0.3 g of supported gold nano-bimetallic catalyst, 5ml of cyclohexane, 25 ml of acetonitrile as solvent and 0.1 g of tert-butyl hydroperoxide (TBHP). These components were placed into an autoclave and flushed three times with O₂ before setting the initial reaction pressure of O₂ to 10 bar. As regards to the beginning of the procedure, the steps were performed with the O₂ line opened, and as O₂ was consumed, it was replaced from the cylinder, which maintains the overall pressure constant. The stirring speed of reaction mixture was set to 1500 rpm in general and the reaction was performed at 150° C. for 4 h unless otherwise stated. At the end of the reaction, the solid bimetallic catalyst was separated by centrifugation. In addition, this reaction was also performed using a glass reactor consisting of 50 ml round-bottomed flask with a reflux-cooling condenser. The reaction conditions used for glass reactor tests were similar to the ones performed in the autoclave except the pressure was ambient. Experiments were carried out using an oil bath at 150° C. for 4 h with continuous air bubbling through the reaction mixture (i.e. in the reactor). At the end of the reaction, the solid bimetallic catalyst was separated by centrifugation. The identity of the reaction products was confirmed by gas chromatography (Agilent 6890 N) fitted with an HP-5 column and a flame ionization detector (FID). In order to obtain the acids in the ester form, 500 μl of product sample was esterified with 400 μl of trimethylsulfonium hydroxide in the presence of internal standard (3-pentanone, 100 μl). After such derivatisation of acid to ester, 0.2 μl of this sample was injected off-line into GC and analyzed.

A blank experiment was also executed by treating CH with oxygen and TBHP at 150° C. in the absence of bimetallic catalyst. This blank test showed a conversion of CH of approximately 0.4% in the first 1 h. Subsequently, the conversion increased gradually to ca. 2 % after 4 hours on-stream. Comparing this result with that of a bimetallic catalyzed reaction, the blank test has exhibited only a very low and negligible conversion and hence presence of a bimetallic catalyst was essential and played a key role on the performance.

The conversion of CH and selectivity of products during the catalytic oxidation reaction were calculated according to the instant disclosure. The primary objective of this study was first to investigate the influence of second-metallic element (i.e. bimetallic system) in the catalytic performance for the direct oxidation reaction of cyclohexane. With these objectives, the following catalysts were prepared according to the procedure and tested as described above and the results are given in Table 1. These catalysts are shown as bare TiO₂ support, monometallic catalyst (e.g. 1% Au/TiO₂ and 1% Pd/TiO₂) and bimetallic catalyst (1% Au-1% Pd/TiO₂). The results of these investigations are displayed in Table 1. The bare TiO₂ was found to show the poorest performance, while the Au/TiO₂ displayed improved catalytic activity (i.e. X-CH=25%, and S-AA=26%). In addition, the monometallic Pd catalyst (i.e. Pd/TiO₂) exhibited somewhat inferior performance (X-CH=16%, S-AA=18%) compared to Au/TiO₂ solid. However, the combination of Au and Pd (AuPd/TiO₂) markedly improved the selectivity of AA from 26% to 34%, which is almost double to the S-AA obtained on Pd/TiO₂ and also remarkably higher even to compared to monometallic Au/TiO₂ catalyst. However, the conversion of CH (X=21%) obtained on bimetallic AuPd /TiO₂ is significantly higher than monometallic Pd/TiO₂ but slightly lesser than monometallic Au/TiO₂ sample. Considering all these effects, it can be claimed that the addition of a second metallic component to the catalyst had a clear promotional effect on the selectivity of AA, which might be due to expected synergistic effects between Pd and Au.

TABLE 1
The catalytic activity of the mono-metallic (Au, Pd) and bi-metallic
(Au—Pd) catalysts in the direct oxidation of cyclohexane:

| | | X-CH | S-AA | S-One | S-Ol | S-others |
|---|---|---|---|---|---|---|
| S. No. | Catalyst* | (%) | (%) | (%) | (%) | (%) |
| 1 | TiO₂ | 5 | 0 | 13.1 | 12.3 | 21.8 |
| 2 | Au/TiO₂ | 26 | 26.3 | 29.3 | 47.1 | 2 |
| 3 | Pd/TiO₂ | 13 | 18 | 12 | 46 | 26.2 |
| 4 | AuPd/TiO₂ | 21 | 34.5 | 26.3 | 37.4 | 1.8 |

X-CH = conversion of cyclohexane; S-AA = selectivity of adipic acid; S-One = selectivity of cyclohexanone; S-Ol = selectivity of cyclohexanol; S-Others = selectivity of glutaric acid, succinic acid, cyclohexylhydroperoxide, CO and CO₂, Reaction conditions: 5 ml CH, 25 ml solvent (acetonitrile), 0.3 g catalyst, 0.1 g TBHP, pO₂ = 10 bar, t = 4 h, 1500 rpm, T = 130° C. (*Au and Pd loading are 1 wt % each).

In addition to Pd as second metal, some tests were also performed using Ag as a second metal. Example below demonstrates the effect of Ag metal on the performance of 1 wt %Au/TiO₂ solid. This means the bi-metallic system here is Au—Ag/TiO₂. The influence of monometallic (1% Ag) and bi-metallic catalysts in the cyclohexane oxidation is shown in Table 2. The catalytic activity of both pure support and Au/TiO₂ were already discussed earlier. The reaction performed using 1% Ag/TiO₂ catalyst has no appreciable influence on the activity and selectivity behaviour compared to Au/TiO₂. Nevertheless, the catalytic performance of using Au—Ag/TiO₂ suggest that the presence of Ag has a clear influence on the performance but in different direction compared to Pd. Using Ag as a second metal, not only the selectivity of KA (S=82%) was significantly improved compared to all mono-metallics (Ag/TiO₂, Pd/TiO₂ and Au/TiO₂) but also the conversion of CH increased. Nevertheless, the influence of Ag addition on the selectivity of AA was not that bad (S-AA=14.3%), which led to a yield of ca. 4%. Considering the effects on the whole, it can be stated that both these metallic components (Pd, Ag) have shown different influences and thereby different distribution of products.

TABLE 2
The catalytic activity of the mono-metallic (Au, Ag) and bi-
metallic (Au—Ag) catalysts on the oxidation of cyclohexane.

| | | X-CH | S-AA | S-One | S-Ol | S-others |
|---|---|---|---|---|---|---|
| S. No. | Catalyst* | (%) | (%) | (%) | (%) | (%) |
| 1 | TiO₂ | 5 | 0 | 13.1 | 12.3 | 21.8 |
| 2 | Au/TiO₂ | 26 | 26.3 | 29.3 | 47.1 | 2 |
| 3 | Ag/TiO₂ | 8 | 10 | 12 | 56.5 | 26.2 |
| 4 | AuAg/TiO₂ | 29 | 14.3 | 36.2 | 46.5 | 10.0 |

X-CH = conversion of cyclohexane; S-AA = selectivity of adipic acid; S-One = selectivity of cyclohexanone; S-Ol = selectivity of cyclohexanol; S-Others = selectivity of glutaric acid, succinic acid, cyclohexylhydroperoxide, CO and CO₂, Reaction conditions: 5 ml CH, 25 ml solvent (acetonitrile), 0.3 g catalyst, 0.1 g TBHP, pO₂ = 10 bar, t = 4 h, 1500 rpm, T = 130° C. (*Au and Ag loading are 1 wt % each).

The foregoing examples have been provided for the purpose of explanation and should not be construed as limiting the present disclosure. While the present disclosure has been described with reference to an exemplary embodiment, changes may be made within the purview of the appended claims, without departing from the scope and spirit of the present disclosure in its aspects. Also, although the present disclosure has been described herein with reference to particular materials and embodiments, the present disclosure is not intended to be limited to the particulars disclosed herein; rather, the present disclosure extends to all functionally equivalent structures, methods and uses, such as are within the scope of the instant claims. Accordingly, the specification and drawings are to be regarded in an illustrative rather than in a restrictive sense.

What is claimed is:

1. A nano-bimetallic catalyst, comprising:
a coinage metal group;
a platinum metal group; and
at least one of a transition metal and a rare earth metal oxide in powder form for support to prepare adipic acid from cyclohexane.

2. The nano-bimetallic catalyst of claim 1, wherein the coinage group of metal is at least one of copper (Cu), silver (Ag), or gold (Au).

3. The nano-bimetallic catalyst of claim 2, wherein the coinage group of metal is at least one of the silver and gold.

4. The nano-bimetallic catalyst of claim 1, wherein the platinum group of metal is at least one of ruthenium, rhodium, palladium, osmium, iridium, and platinum.

5. The nano-bimetallic catalyst of claim 4, wherein the platinum group of metal is palladium.

6. The nano-bimetallic catalyst of claim 1, wherein the support is a transition metal oxide, wherein the transition metal is titanium.

7. A method of making the nano-bimetallic catalyst, comprising:
dissolving tetracholoroauric acid and potassium carbonate in distilled water to make a first solution;
dissolving a platinum group of metal chloride in water to make a second solution;
heating the second solution and adding hydrochloric acid to dissolve the platinum group of metal chloride in the second solution completely;
adding the first solution to the second solution to make a third solution wherein the third solution is a bimetallic solution and
adding the third solution to a support to form a colloidal solution, wherein the support is titanium oxide.

8. The method of making the nano-bimetallic catalyst as in claim 7, wherein the platinum group of metal chloride is palladium chloride.

9. (canceled)

10. The method of making the nano-bimetallic catalyst as in claim 7, further comprising;
reducing the bimetallic solution using a reducing agent in a solvent to produce a stabilized bimetallic solution; and
stabilizing the reduced bimetallic solution by stirring with a stabilizer to obtain a colloidal bimetallic solution.

11. The method of making the nano-bimetallic catalyst as in claim 10, wherein the reducing agent is at least one of citric acid, sodium citrate, ascorbic acid, sodium thiocyanate, sodium borohydride, tannic acid, tartaric acid and oxalic acid.

12. The method of making the nano-bimetallic catalyst as in claim 10, further comprising:
making a slurry from combining a catalyst support and the colloidal bimetallic solution to impregnate the bimetal colloid into the support;
removing an excess solvent from the slurry by using rotary evaporation to make a solid bimetallic catalyst; and
calcinating in a specific atmosphere and oven drying the solid bimetallic catalyst to form a nano-bimetallic catalyst.

13. The method of making the nano-bimetallic catalyst as in claim 12, wherein the nano-bimetallic catalyst is AuPd/TiO₂ or AuAg/TiO₂ catalyst.

14. The method of making the nano-bimetallic catalyst as in claim 12, wherein the specific atmosphere is at least one of an inert gas, air and reducing gas.

15. A method of making adipic acid(AA), comprising:
oxidizing a cyclic paraffin mixed with a solvent using a gas, an inert atmosphere and a nano-bimetallic catalyst in an autoclave; and
applying a specific pressure and a specific temperature to the autoclave while oxidizing the cyclic paraffin to make adipic acid.

16. The method of making adipic acid(AA) as in claim 15, wherein the cyclic paraffin is cyclohexane.

17. The method of making adipic acid(AA) as in claim 15, wherein the gas is at least one of oxygen, air and mixture of oxygen and nitrogen.

18. The method of making adipic acid(AA) as in claim 15, wherein the nano-bimetallic catalyst is AuPd/TiO₂ catalyst.

19. The method of making adipic acid(AA) as in claim 15, wherein the temperature is between 50-250° C.

20. The method of making adipic acid(AA) as in claim 15, wherein the specific pressure is at least one of atmospheric, sub-atmospheric and super-atmospheric.
